(19) United States
(12) Patent Application Publication
(10) Pub. No.: US 2026/0330953 A1
(43) Pub. Date: Oct. 22, 2026

(54) System and Method for Managing a Creative Nap

(71) Applicant: Claude Coulombe, Montreal (CA)

(72) Inventor: Claude Coulombe, Montreal (CA)

(21) Appl. No.: 19/119,268

(22) PCT Filed: Oct. 18, 2023
(86) PCT No.: PCT/CA2023/051377

§ 371 Date: Apr. 8, 2025

(51) Int. Cl. G08B 21/06; G10L 25/66

(52) U.S. Cl. CPC G08B 21/06; G10L 25/66

(57) ABSTRACT

A computer-implemented method is described for managing a creative nap, and includes using a processor located on a computing device to initiate a number of method steps. At the computing device, receiving sound of a gadget hitting the floor, one or more signals wirelessly transmitted thereto from a handheld sensor. The one or more signals are transmitted to a user when the handheld signal falls away from the user. Transmitting a triggering signal from the sensor to the computing device when the sensor is detected at a predetermined distance away from the computing device. Triggering a waking alarm to wake the user within a predetermined time; and then prompting the user to record and store verbal communications in the mobile electronic device as an idea data package.

### TECHNICAL FIELD

The present generally concerns sleep management, and more particularly to management of creative thinking during early sleep phase.

### BACKGROUND

It is well known that waking up immediately before falling asleep can help a person improve their creative thinking. Indeed, recent scientific studies have demonstrated that humans have a brief period of creativity when they start to fall asleep (see for example “Sleep onset is a creative sweet spot” in Science Advances, Dec. 8, 2021 to Lacaux et al.). This is called the Ni phase of sleep, also known as “non-rapid eye movement sleep stage 1”.

A number of groups have attempted to exploit electronic devices in the management of sleep cycles and data collected from a sleeping subject. The following examples are available:
WO 2019/213804 for “Method for Operating an Electronic Device as an Alarm Clock” to Orange et al; and
U.S. Pat. No. 10,791,986 for Sleep Sound Detection System and Use to Kahn, Philippe et al.

The WO 2019/213804 publication discloses an electronic device, such as a smartphone, which is connected to a distinct sensor for monitoring a physiological parameter of a user. One such sensor is an accelerometer for measuring the user's body movement to detect a sleep state. However, disadvantageously the accelerometer is in fixably connected to the user's body, which may restrict its usefulness, especially if the user attempts to move the accelerometer away from the body. Also, during operation, an alarm signal is provided after a desired sleep time elapses, and not immediately upon detection of the sleep state, so as to interrupt or break the sleep while it is still light.

The U.S. Pat. No. 10,791,986 system and device monitor a user's sleep and turns on a microphone to record sounds. It also discloses a motion sensor, which is believed to be an accelerometer, so as to detect the user's sleep state. However, disadvantageously the device in the U.S. Pat. No. 10,791,986 captures and records sleeping sounds while the user is asleep. is notably different from your invention. Furthermore, like the WO 2019/213804 system, the motion sensor is in fixed relation to the user's body.

Thus, there is clearly an unmet need for a method and system that includes a sensor and a method of recording a user's input in the form of speech.

### BRIEF SUMMARY

We have significantly reduced, or essentially eliminated, the problems associated with the designs described above by designing a system and method for managing a creative nap. The system includes hardware components of an electronic computing device, for example a mobile computing device, and a peripheral device in the form of a sensor that is communicatively coupled with the computing device. In the examples illustrated herein, the electronic computing device is a smartphone or a tablet. In other examples, the computing device can also include a laptop or a desktop with a Bluetooth I/O or other appropriate wifi communication mean. Advantageously, the system is physically detached therefrom, so as to be able to move relative thereto. More specifically, the sensor is configured to be held in the hand and is weighted to be configured to fall, or be gravitationally displaced, when freed from the user's hand at the beginning of nap onset. The system operates to record creative information in the form of speech, voice comments, and the like, or data of the user by (i) monitoring a user's sleep state, which is achieved by detecting the sound of a gadget when hitting the floor after it falls out of the user's hand, and by measuring a distance between the sensor and the smartphone, (ii) when the sleep state is detected. or after a parameterized time delay, which can be fixed using machine-learning on the user's data, generating a signal to wake the user, and (iii) requesting the user's input as to their thoughts and recording the user's speech on the smartphone. Furthermore, the system can be adapted in a parameterized manner. In this case, if the user is too sleepy or fails to hear the sound of the gadget hitting the floor, the user may wish to delay waking until after a few second or minutes. Finally, from an economic point of view, our system uses a beacon (a Bluetooth device) which is less expensive than a combined accelerometer plus a Bluetooth device.

Accordingly, there is provided an embodiment in which a computer-implemented method for managing a creative nap, comprising using a processor located on a computing device to initiate the following method steps:
at the computing device, receiving one or more signals wirelessly transmitted thereto from a handheld sensor, the one or more signals being transmitted to a user when the handheld signal falls away from the user;
transmitting a triggering signal from the sensor to the computing device when the sensor is detected at a predetermined first parameter, the first parameter being calculated relative to the computing device;
triggering a waking alarm to wake the user within a predetermined second parameter;
triggering a waking alarm to wake the user within a predetermined third parameter; and
prompting the user to record and store communications in the mobile electronic device as an idea data package. The user can optionlly enter text directly.

In one example, the first parameter is a calculated predetermined distance between the sensor and the computing device.

In one example, the first parameter is a detectable signal strength, signal level or signal intensity.

In one example, the first parameter is the detection of the noise when a gadget contacts the floor. A Bluetooth beacon triggered in the event the noise signal is not detected or if the user is too sleepy or did not hear the sound of the gadget hitting the floor or maybe the user does not want to be wake up immediately but only after few seconds or minutes.

In one example, the computing device further includes a noise detection mechanism having a microphone adapted to be triggered for a noise signal on the device triggering for specific sound (gadget hitting the floor).

In one example, the method further includes an optional machine learning system adapted to be calibrated and trained during a user experimental session, the session being managed to train the user on different sounds and physical distance including noise of a gadget hitting the floor, the system being further calibrated by the user as needed.

In another example, the predetermined second parameter is time.

In another example, the predetermined second parameter is optimized by machine learning based on a user's experimental data.

In yet another example, the communications are verbal communications.

In another example, the method further includes: calculating a distance, a signal strength, a signal level or a signal intensity between the computing device and the sensor so that when the distance is greater than a predetermined distance, or the signal strength is below a threshold, the waking alarm triggers.

In one example, the predetermined threshold distance is about 50 cm.

In one example, the one or more signals are analyzed to determine the speed of the sensor distance change relative to the computing device, taking into account the predetermined time.

In one example, the computing device includes a Bluetooth transmitter/receiver.

In one example, the hand-held sensor includes a Bluetooth transmitter beacon.

In one example, the waking alarm is an audible signal. The audible signal is repeated. In one example, a voice prompt requests the user to input the voice recording.

In one example, if desired, the user stops the recording by activating a button on a phone screen interface.

In yet another example, a voice transcription option permits the user to transcribe the voice recording into text

In one example, the computing device is a handheld computing device. The handheld computing device is a mobile phone.

Accordingly in another embodiment there is provided a system for managing a creative nap, the system comprising:
a computing device;
a sensor in wireless communication with the computing device and remotely located therefrom and moveable relative to the computing device, the sensor being sized and shaped to be held in a hand of a user, the sensor being of a sufficient weight such that when displaced from the user's hand, the sensor moves towards the ground;
a data recorder located in the computing device and in electronic communication with the sensor, the data recorder being operable to collect a set of sleep state data, the sleep state data being collected by measuring a distance or a signal strength variation between the sensor and the computing device;
a signal transmitter for sending a triggering signal from the handheld sensor to the computing device when the sensor is detected at a predetermined distance or a signal level away from the computing device;
an alarm to wake the user within a predetermined parameter so as to prompt the user to begin recording; and
a data recorder for collecting and storing verbal communications in the electronic device, the data recorder being activated after the waking alarm prompts the user to begin recording.

In one example, the first parameter is a calculated predetermined distance between the sensor and the computing device.

In one example, the first parameter is a detectable signal strength, signal level or signal intensity.

In one example, the first parameter is the detection of the noise when a gadget contacts the floor. A Bluetooth beacon triggered in the event the noise signal is not detected or if the user is too sleepy or did not hear the sound of the gadget hitting the floor or maybe the user does not want to be wake up immediately but only after few seconds or minutes.

In one example, the computing device further includes i) a noise detection mechanism having a microphone adapted to be triggered for a noise signal on the device triggering for specific sound (gadget hitting the floor).

In one example, the system further includes an optional machine learning system adapted to be trained during a user experimental session, the session being managed to train the user on different sounds including noise of a gadget hitting the floor, the system being further optimized by the user as needed.

In one example, the predetermined second parameter is time.

In one example, the predetermined second parameter is optimized by machine learning based on a user's experimental data.

In one example, the communications are verbal communications.

In one example, the system further includes: calculating a distance, a signal strength, a signal level or a signal intensity between the computing device and the sensor so that when the distance is greater than a predetermined distance, or the signal strength is below a threshold, the waking alarm triggers.

In one example, the predetermined threshold distance is about 50 cm, but can be calibrated and fine-tuned by the user.

In one example, the one or more signals are analyzed to determine the speed of the sensor distance change relative to the computing device, taking into account the predetermined time.

In one example, the computing device includes a Bluetooth transmitter/receiver.

In one example, the hand-held sensor includes a Bluetooth transmitter beacon.

In one example, the waking alarm is an audible signal. The audible signal is repeated. In another example, a voice prompt requests the user to input the voice recording.

In yet another example, if desired, the user stops the recording by activating a button on a phone screen interface.

In still another example, a voice transcription option permits the user to transcribe the voice recording into text In one example, the computing device is a handheld computing device. The handheld computing device is a mobile phone.

### BRIEF DESCRIPTION OF THE DRAWINGS

These and other features of that described herein will become more apparent from the following description in which reference is made to the appended drawings wherein: FIG. 1 is a diagrammatic representation of the connectivity between a user's hand-held mobile computing device and a hand-held sensor;

FIG. 2 is a diagrammatic representation of a computer system;

FIG. 3 is a block diagram showing the steps of the software application;

FIG. 4 is a screenshot of a mobile application showing a touch screen icon used to initiate the system;

FIG. 5 is a photograph of an “egg-shaped” connector object having a sensor located therein (not shown); and

FIG. 6 is a photograph of a disassembled connector object in which the sensor components are displayed.

### DETAILED DESCRIPTION

### Definitions

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus, the phrase “consisting of” indicates that the listed elements are required or mandatory and that no other elements may be present.

Generally speaking, our system permits the management of a creative nap in a human subject by monitoring the subject's sleep patterns. Our system is referred herein as EurekIA. The system includes a smart phone, a mobile app; a sensor that is configured to monitor the user's noise input while falling asleep; and an alarm signal so that the user upon waking is able to collect and record ideas, comments and reflections expressed vocally about a problem, a situation, a work of art and the like, which the user thought about before falling asleep and during the N1 phase of sleep. Optionally, or additionally, noise detection of the gadget (see below) hitting (or contacting) the floor together with the use of Bluetooth, in case the system fails to detect the noise or the sound of the gadget noise falling out of the user's hand when hitting the floor.

A person of ordinary skill in the art will readily understand that although a single hand-held computing device is illustrated, two or more computing devices that communicate over any type of communications link, such as a network, a shared memory, or the like, can perform the method steps described herein. Furthermore, while performing the process or method steps described herein, one or more computing devices can communicate with one or more additional computing devices that are external to the computer infrastructure using any type of communications link. The communications link includes any combination of wired and/or wireless links; any combination of one or more types of networks, for example, the Internet, a wide area network, a local area network, a virtual private network and the like; and/or utilize any combination of transmission techniques and protocols.

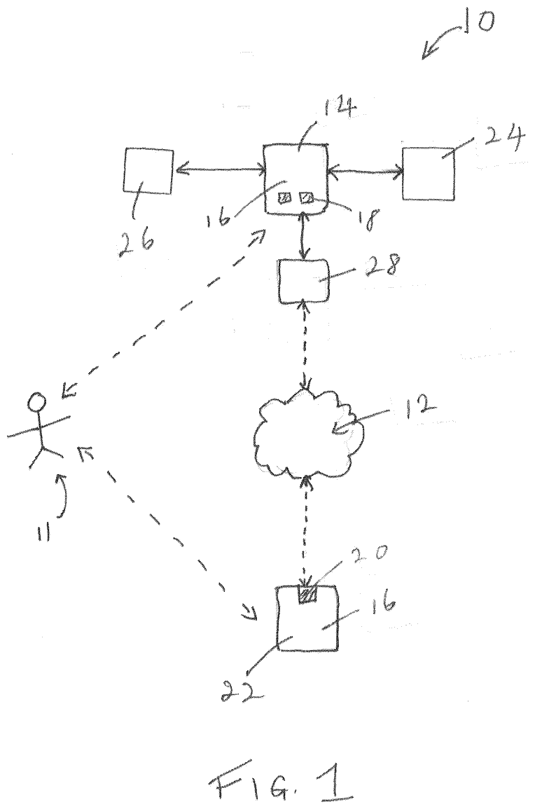

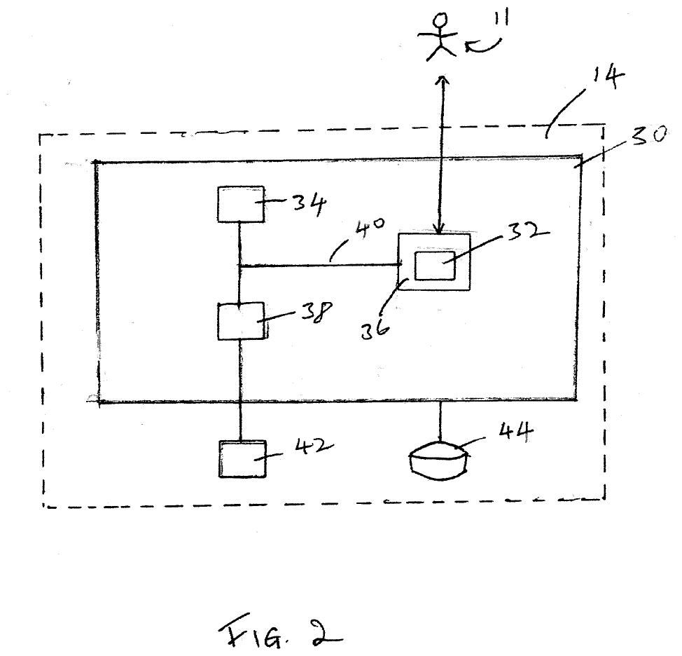

Referring now to FIGS. 1 and 2, our system is shown generally at 10 for use by a subject 11. In the example illustrated, the subject is a human subject. The system 10 includes a network 12 in which wireless communication interconnects the user 11 with a mobile computing device 14 and a hand-held sensor 16. In the example shown, the mobile computing device 14 is a hand-held mobile phone such as a smartphone (iPhone, Android and the like), or a Tablet.

The user 11 has access to information on the system via the smartphone 14. Typically, the smartphone grants access to the system by way of Wi-Fi or Bluetooth connection. A display screen 16 includes a plurality of icons 18 that are activated by touchscreen technology. At the beginning of the process, the user 11 encounters an application which they open by interacting with a Graphical User Interface (GUI) on the display screen 16 on the smartphone 14.

More specifically now, and with reference to FIGS. 1 through 3, the system 10 is part of the wireless network 12 which includes the mobile computing device 14, and the handheld sensor 16. In the example shown, the sensor 16 includes a Bluetooth beacon 20 that is integrated into a hand-held gadget 22 that the user 11 holds in a hand. When the user 11 falls asleep, generally speaking, the muscles relax and the hand opens causing the gadget 22 to fall towards the ground. We designed the gadget 22 to be sufficiently large and heavy so that it slips out of the hand when the muscles relax when the user 11 is falling asleep. Several examples of shaped gadgets which we have designed include the gadget in the form of a lightbulb in homage to the inventor Thomas Edison. Similarly, other designs include Salvador Dali's “soft watch”, Joseph Armand-Bombardier's ball bearing and the like. In each example, the gadget has a weight of about 300 g. Also, the gadget can be equipped with a sleigh bell to provide more noise. The gadget or the bell may be cushioned by use of fabric or a cushion to block the bell noise, as needed by the user.

To detect falling asleep, the software application is based on 1) noise detection and 2) the calculated distance between the smartphone 14 that is equipped with a Bluetooth transmitter/receiver 24 and the gadget 22 that is equipped with the Bluetooth beacon 20. The two mechanisms can be both active or each one separately activated by a parameter of the software application. When the distance is suddenly greater than a threshold, say 50 cm, this is a signal that the gadget 22 had fallen from the user's hand and has just fallen asleep. The software that analyzes the signal takes into account the speed of the position change.

At this time, an audible signal from a sound transmitter 26 such as a Beep or pre-selected music (user selected) will sound a number of times. The user typically selects the wake-up cycle, and the time of the wake-up cycle. At the audible signal, a voice is heard asking the user to record his ideas and then activates a voice recording function 28. At any time, the user can take control of the software by pressing a button on the phone screen 16.

An enhanced version of the software provides voice commands prefixed with a wake word (Hey Eurekia!) and a voice transcription option that uses artificial intelligence to transcribe voice recording into text (audio-to-text transcription) Referring first to FIG. 2, the smartphone 14 includes a computing system 30 with a sleep management system 32, which allows the smartphone 14 to perform management of creative naps according to the method steps described below. The computing device 14 includes a processor 34, a non-transitory memory 36, an input/output (1/O) interface 38, and a bus 40. Further, the computing system 30 is in communication with, if necessary, an external I/O device/resource 42, and a data storage 44.

Generally speaking, the processor 34 executes computer program code, which is stored in the memory 36 and/or the data storage 44. While executing computer program code, the processor 34 can read and/or write data to/from the memory 36, the data storage 44, and/or I/O interface 42. The bus 40 provides a communications link between each of the components in the computing system 30. The I/O device 42 may include a device that permits the user to interact with the computing system 30 or a device that permits the computing system 30 to communicate with one or more other computing devices using any type of communications link known to those skilled in the art.

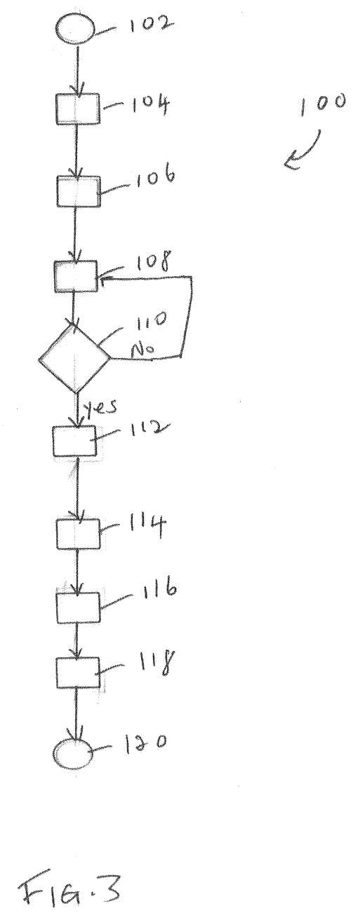

Referring now to FIG. 3, a block diagram 100 generally illustrates the steps included in the method. At block 102, the user begins the software application by opening the app. At this point, at block 104, the user then sets the awake time and signal strength or distance threshold between the smartphone and the sensor. At block 106, the sensor is then activated. While the user begins to fall asleep the monitoring begins at block 108. If, at block 110, the user continues to fall asleep and the sensor remains in his hand, the cycle returns back to block 108. If however, the sensor detects falls at block 112, the distance is analyzed and if it exceeds the threshold at block 104, block 116 then issues an alarm signal to awake the user. At block 118, the user is then prompted to record his ideas and the like. Once completed, the user then presses a stop button to cease recording and at block 120, the program finishes.

A person of ordinary skill in the art will recognize that other forms of sensor signal may be used. For example, one or more signals may be transmitted in the form of sound (a filtered sound which removes background noise), a light signal together with a vibration detector.

Additionally, material surrounding the sensor may be a foam or cushioned material of sufficient resilience such that the sound of the sensor hitting the floor doesn't wake the user. Instead, the sensation of the sensor hitting the ground triggers the sensor (after a predetermined time) to transmit a wake signal to the user's device.

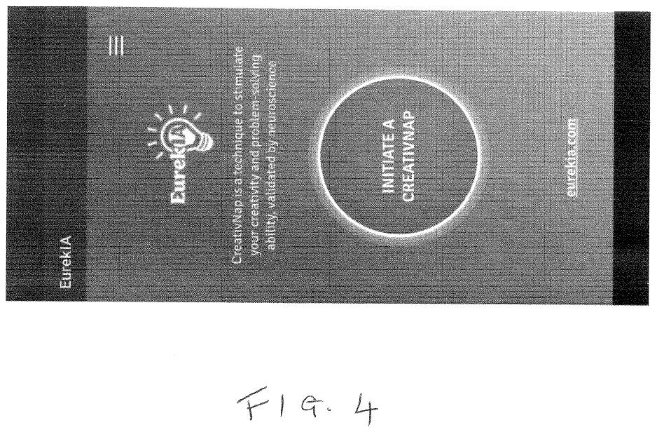

Referring now to FIGS. 4 to 6, in which generally speaking, the sounds detected by the deep neural network (the so-called “convolutional network”) can be referred to as a “tinkling” sound. However, a person skilled in the art may also recognize that the sound could be likened to any bell, sleigh bell, hand bell or the sound of a kitchen utensil against a hard object, like a spoon falling on a plate.

### 1) Mobile App

We have developed and programmed two versions (Android and iOS) of the EurekIA mobile phone application have been programmed. As best seen in FIG. 4, the screenshot of the mobile app offers a user interface in order to 1) record and manage audio recordings of the ideas; 2) select between two falling asleep detection mechanisms: bell sound recognition and Bluetooth signal analysis; and 3) tune the sensor parameters, phone ringing parameters, duration, delay before activating phone ringing, voice over explanations and the like.

### 2) Algorithms

The falling asleep detection algorithm includes two main methods of detection:
i) detection of a ringing (bell sound) thanks to a sound recognition algorithm based on deep learning. Specifically, a convolutional network; and
ii) detection of the distance of the Bluetooth beacon from the connected object, via an automatic learning algorithm inspired by the breakpoint detection algorithm and the cumulative sum algorithm (CUSUM).

Alternatively, the fall may be detected using a beacon that contains an accelerometer.

### 3) Connected Object/Sensor

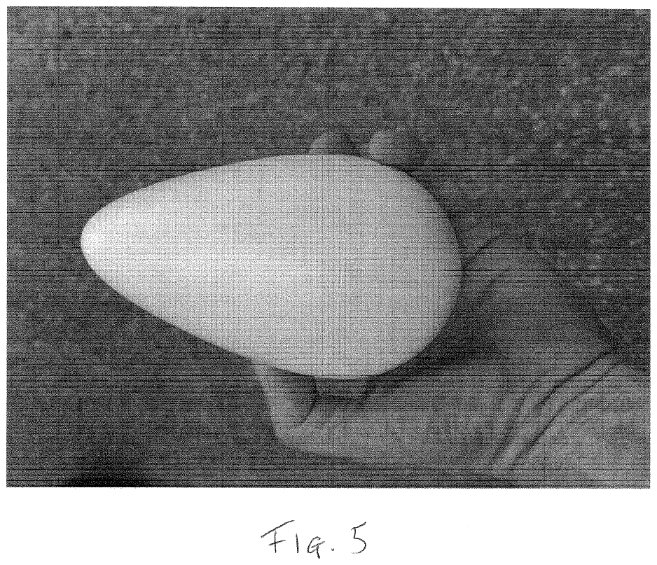

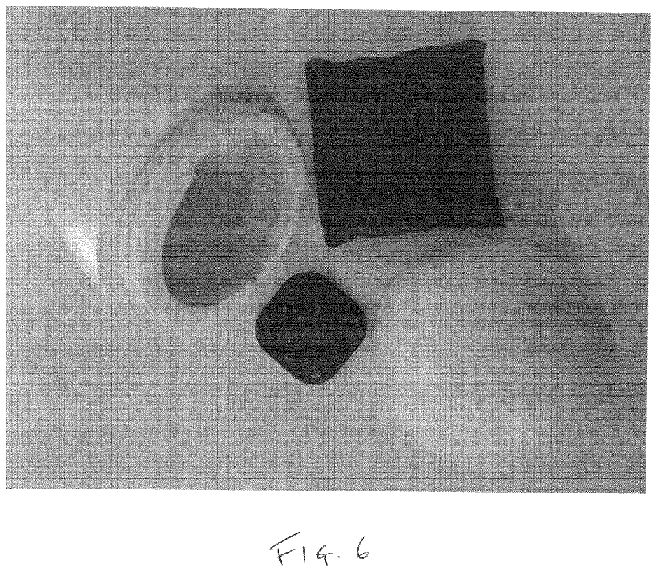

Referring now to FIGS. 5 and 6, for the connected object, which contains a Bluetooth beacon, we designed a shaped object, specifically in the form of a pyriform/pear-shaped egg. We labeled this a “EurekIA egg”. For both aesthetic and practical purposes, the desired shaped was chosen because it mimics the razorbill bird (Latin: Alca torda) which never rolls too far from its point of fall. Indeed, after a fall, the eggs turn on its base thus avoiding falling over a cliff. A person skilled in the art will recognize that other shapes are permissible, such as a connected object in the form of a light bulb.

### Other Embodiments

From the foregoing description, it will be apparent to one of ordinary skill in the art that variations and modifications may be made to the embodiments described herein to adapt it to various usages and conditions.

1. to 22. (canceled)

23. A system for managing a creative nap, the system comprising:
a computing device;
a sensor in wireless communication with the computing device and remotely located therefrom and moveable relative to the computing device, the sensor being sized and shaped to be held in a hand of a user, the sensor being of a sufficient weight such that when displaced from the user's hand, the sensor moves towards the ground;
a data recorder located in the computing device and in electronic communication with the sensor, the data recorder being operable to collect a set of sleep state data, the sleep state data being collected by measuring a distance or a signal strength variation between the sensor and the computing device;
a signal transmitter for sending a triggering signal from the handheld sensor to the computing device when the sensor is detected at a predetermined distance or a signal level away from the computing device;
an alarm to wake the user within a predetermined parameter so as to prompt the user to begin recording; and
a data recorder for collecting and storing verbal communications in the electronic device, the data recorder being activated after the waking alarm prompts the user to begin recording.

24. The system, according to claim 23, in which the first parameter is a calculated predetermined distance between the sensor and the computing device.

25. The system, according to claim 23, in which the first parameter is a detectable signal strength, signal level or signal intensity.

26. The system, according to claim 23, in which the first parameter is the detection of the noise when a gadget contacts the floor.

27. The system, according to claim 26, in which a Bluetooth beacon triggered in the event the noise signal is not detected or if the user is too sleepy or did not hear the sound of the gadget hitting the floor or maybe the user does not want to be wake up immediately but only after few seconds or minutes.

28. The system, according to claim 23, in which the computing device further includes i) a noise detection mechanism having a microphone adapted to be triggered for a noise signal on the device triggering for specific sound (gadget hitting the floor).

29. The system, according to claim 23, further includes an optional machine learning system adapted to be trained during a user experimental session, the session being managed to train the user on different sounds including noise of a gadget hitting the floor, the system being further optimized by the user as needed.

30. The system, according to claim 23, in which the predetermined second parameter is time.

31. The system, according to claim 23, in which the predetermined second parameter is optimized by machine learning based on a user's experimental data.

32. The system, according to claim 23, in which the communications are verbal communications.

33. The system, according to claim 23, further includes: calculating a distance, a signal strength, a signal level or a signal intensity between the computing device and the sensor so that when the distance is greater than a predetermined distance, or the signal strength is below a threshold, the waking alarm triggers.

34. The system, according to claim 23, in which the predetermined threshold distance is about 50 cm, but can be calibrated and fine-tuned by the user ..

35. The system, according to claim 23, in which the one or more signals are analyzed to determine the speed of the sensor distance change relative to the computing device, taking into account the predetermined time.

36. The system, according to claim 23, in which the computing device includes a Bluetooth transmitter/receiver.

37. The system, according to claim 23, in which the hand-held sensor includes a Bluetooth transmitter beacon.

38. The system, according to claim 23, in which the waking alarm is an audible signal.

39. The system, according to claim 38, the audible signal is repeated.

40. The system, according to claim 23, in which a voice prompt requests the user to input the voice recording.

41. The system, according to claim 23, if desired, the user stops the recording by activating a button on a phone screen interface.

42. The system, according to claim 23, a voice transcription option permits the user to transcribe the voice recording into text

43. (canceled)

44. (canceled)
